(19) United States
(12) Patent Application Publication
(10) Pub. No.: US 2026/0048196 A1
(43) Pub. Date: Feb. 19, 2026

(54) DEVICE FOR DELIVERING INSULIN HAVING FILM AND TOP ENCLOSURE DEFINING RESERVOIR

(71) Applicant: AITA BIO INC., New York, NY (US)

(72) Inventor: David Coleman, Dublin (IE)

(21) Appl. No.: 19/296,045

(22) Filed: Aug. 11, 2025

Related U.S. Application Data
(60) Provisional application No. 63/682,948, filed on Aug. 14, 2024.

(51) Int. Cl. A61M 5/142

(52) U.S. Cl. CPC A61M 5/14248

(57) ABSTRACT

A device is disclosed for delivering medication to a user. The device comprises first and second enclosures that together form a housing of the device, the housing configured to house a reservoir for storing the medication, wherein the first enclosure includes an inner wall of the housing; and a film joined to the inner wall of the first enclosure, wherein the film and the inner wall of the first enclosure are configured to form walls of the reservoir for storing the medication.

### CROSS REFERENCE TO RELATED APPLICATIONS

This application claims priority to U.S. provisional application 63/682,948 filed Aug. 14, 2024, entitled “Device For Delivering Insulin Having Film Material and Top Enclosure Defining Reservoir” which is incorporated by reference herein.

### FIELD OF THE INVENTION

A device for delivering insulin integrates a reservoir into its housing by utilizing a top enclosure as part of the reservoir.

### BACKGROUND OF THE INVENTION

Insulin pumps help people with diabetes to conveniently manage their blood sugar. These devices deliver insulin at specific times. Insulin patch pumps or pods are one type of insulin pump. The patch pumps are wearable devices that adhere to the skin of a user using an adhesive patch. The patch pumps are controlled wirelessly with a handheld controller. The patch pumps deliver insulin from a chamber and internal cannula based on separately acquired CGM sensor readings. The fluidic components within the patch pumps are typically significant in number to achieve proper fluid delivery. Component integration for such pumps are complex and current solutions are thus large and expensive. This complication risks device malfunction.

It would be advantageous to provide improvements to these pumps.

### SUMMARY OF THE INVENTION

A device for delivering insulin includes a top enclosure and film that act as walls of a reservoir.

In accordance with an example of the disclosure, a device for delivering medication to a user, the device comprising first and second enclosures that together form a housing of the device, the housing configured to house a reservoir for storing the medication, wherein the first enclosure includes an inner wall of the housing; and a film joined to the inner wall of the first enclosure, wherein the film and the inner wall of the first enclosure are configured to form walls of the reservoir for storing the medication.

In yet another example of the disclosure, a device for delivering medication to a user, the device comprising: first and second enclosures that together form a housing of the device; a reservoir integrated into the housing configured to store the medication; wherein the first enclosure includes an inner wall and a tray along the inner wall of the first enclosure; and a film joined to the tray, wherein the film and the tray are configured to form walls of the reservoir.

In yet another example of the disclosure, a method of integrating a reservoir into a housing of a device for delivering medication, wherein the housing includes a reservoir section for the reservoir, the method comprising: joining a film to the reservoir section of the housing; and forming the film against a wall of the reservoir section of the housing to thereby remove any air trapped between the film and wall of the reservoir.

### BRIEF DESCRIPTION OF THE DRAWINGS

FIG. 1 depicts perspective view of an example device for delivering insulin or other medication incorporating a housing with a top enclosure and bottom enclosure.

FIG. 2A depicts a bottom perspective view of the top enclosure in FIG. 1 along with a film that together define a reservoir in an unfilled configuration.

FIG. 2B depicts a bottom perspective view of the top enclosure in FIG. 1 along with a film that together define a reservoir in a filled configuration.

FIG. 3 depicts a sectional view of the top enclosure and film of FIG. 2.

FIG. 4 depicts a bottom perspective view of a top enclosure and film of another example device for delivering insulin or other medication, the top enclosure and film defining a reservoir in an unfilled configuration.

FIG. 5 depicts an enlarged sectional view of the top enclosure and film of FIG. 4.

FIG. 6 depicts a cross-sectional view of the device in full, taken along line 6-6 in FIG. 4.

FIG. 7 depicts a cross-sectional view of the device in full, taken along line 6-6 in FIG. 4, with the reservoir in a filled configuration.

FIG. 8 depicts a bottom perspective view of a top enclosure and film of another example device for delivering insulin or other medication, the top enclosure and film defining a reservoir in an unfilled configuration.

FIG. 9 depicts a cross-sectional view of the device in full, taken along line 9-9 in FIG. 8.

FIG. 10 depicts perspective view of another example device for delivering insulin or other medication incorporating a housing with a top enclosure and bottom enclosure.

FIG. 11 depicts perspective view of the device in FIG. 10.

FIG. 12 depicts a bottom perspective view of the top enclosure in FIG. 11 along with a film that together define a reservoir in an unfilled configuration.

FIG. 13 depicts a perspective view of the bottom enclosure in FIG. 10.

FIG. 14 depicts a cross-sectional view of the device taken along line 14-14 in FIG. 12, illustrating the reservoir in an unfilled configuration.

FIG. 15 depicts a cross-sectional view of the device in FIG. 10, illustrating the reservoir in a filled configuration.

FIG. 16 depicts a cross-sectional view of the device in FIG. 10 taken along line 16-16 in FIG. 12, illustrating the reservoir in an unfilled configuration.

FIGS. 17 and 18 depict cross-sectional views of machinery used for joining a film to a top enclosure of a device for delivering insulin.

FIG. 19 depicts a box diagram of components of an example device for delivering medication.

### DETAILED DESCRIPTION OF THE INVENTION

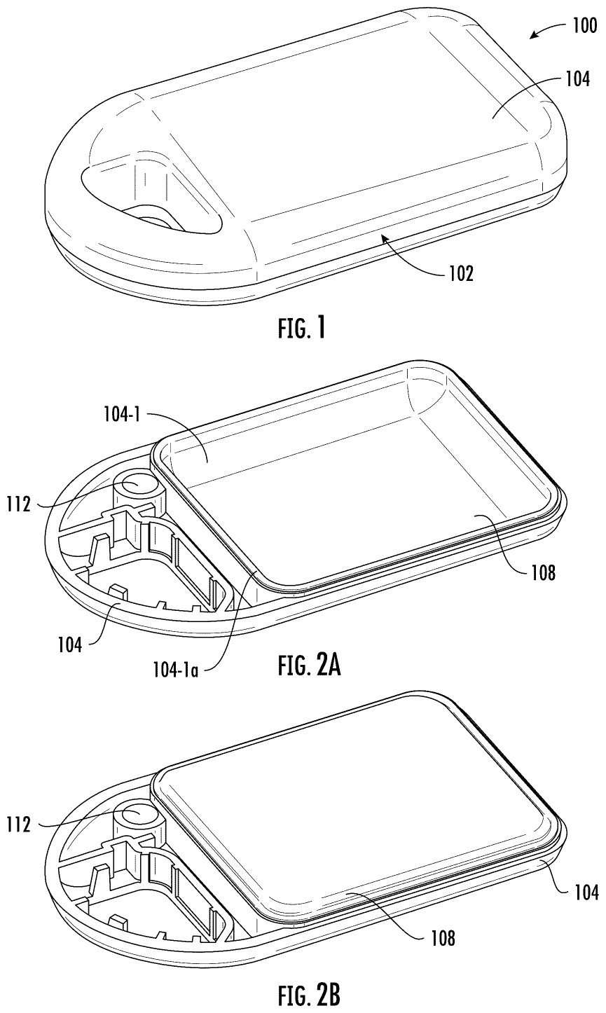

In FIG. 1, example device 100 for delivering insulin is (the device is also referred to as a delivery device or pod). Device 100 includes a reservoir (described below) as well other components such as a micropump or other pump as known to those skilled in the art that can be used for pumping the insulin, valves used for regulating flow, actuators used for moving or controlling the micropump and valves and/or sensors used for sensing pressure and/or flow, microcontroller unit (MCU), battery and power controller, insertion needle(s), infusion catheter and/or CGM sensor as described in more detail below.

The micropump may be used to infuse the insulin or other fluidic medication to the user (patient). Medication may include small molecule pharmaceutical solutions, large molecule or protein drug solutions, saline solutions, blood or other fluids known to those skilled in the art. Insulin is an example of medication or fluid that is described in this application. However, micropump may be used in other environments known to those skilled in the art. The micropump may be a MEMS (micro-electromechanical systems) pump or another pump known to those skilled in the art. The micropump fluidly communicates with a reservoir to enable infusion as needed. CGM, as known to those skilled in the art, tracks patient glucose levels and permits those levels to be used in algorithms that control flow rate.

The MCU is configured to control the operation of the micropump to deliver insulin through the infusion catheter from the reservoir at specific doses, i.e., flow rates over specified time intervals, based on CGM data converted to desired flow rate via control algorithms. The battery and power controller controls the power to the MCU and the micropump to enable those components to function properly as known to those skilled in the art. The CGM is powered by battery and the power controller through the MCU. In short, device 100 integrates a reservoir into its housing by utilizing a top enclosure as part of the reservoir which allows for maximum medication volume fill with a minimum amount of device 100 real estate required.

### Reservoir.

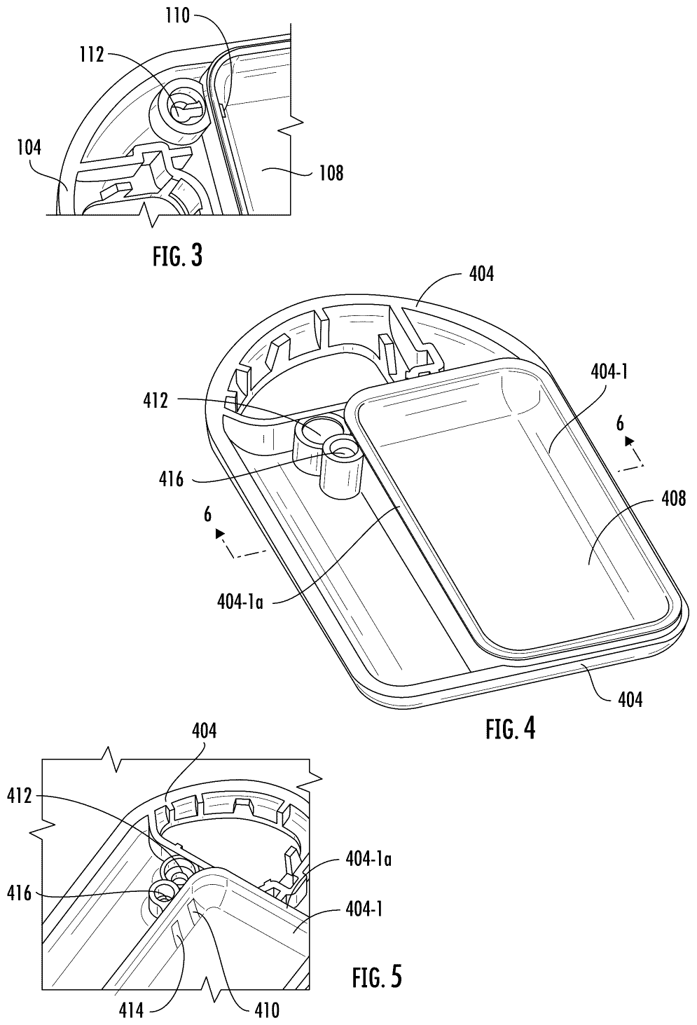

References is made to FIGS. 1-3 wherein device 100 is shown in various views. Device 100 includes housing 102 with top enclosure 104 and bottom enclosure 106. Top enclosure 104 incorporates tray 104-1 along the inner wall of top enclosure 104. Tray 104-1 is essentially the reservoir section of housing 102. Tray 104-1 includes ledge 104-1a that extends around the periphery of tray 104-1. Ledge 104-1a defines the outer contour of an opening of tray 104-1 leading to a trough or cavity within tray 104-1 as shown. In this example device, tray 104-1 is rectangular and extends the entire width of top enclosure 104. However, tray 104-1 may be any size or shape that achieves desired results.

Device 100 further includes a film 108 that is joined to ledge 104-1a using a heat-staking process and then heat formed to the contours of a wall of tray 104-1 that define the trough. That is, film 108 is thus fully line to line formed against the wall which removes any air that may be trapped between film 108 and wall of tray 104-1. Tray 104-1 and film 108 together define a reservoir for receiving and storing insulin or other medication. FIGS. 2A and 2B do depict the reservoir itself, but it is best seen in FIG. 2B and in other figures of different example delivery devices. Tray 104-1 has an opening or hole 110 to enable a user to fill the reservoir and expand film 108 as reservoir is filled. Fill port 112 communicates with hole 110 and such port 112 is used to fill the reservoir through that hole 110. Fill port 112 leakage is contained using a stopper or other mechanism. A syringe (not shown) may be used to puncture (to be inserted into) the stopper to access hole 110 to fill the reservoir.

In this application as defined above, tray 104-1 of top enclosure 104 of the housing 102 is used as a wall (half) of the reservoir. Film 108 (material) is bonded (such as heat staking) to the surface of an inner wall of top enclosure 104. Film 108 (material) effectively forms a pouch with top (upper) enclosure 104. That is, the film (material) and inner wall of top enclosure 104 together define a cavity for storing the insulin that communicates with a septum for filling as described in more detail below. Using top enclosure 104 as part of the reservoir allows for maximum space usage in the housing and utilizes the curvature in top enclosure 104. In this way, the reservoir configuration takes advantage of the geometry of top enclosure 104 of delivery device 100 as one side of the reservoir and a single thin film 108 as the corresponding part. This utilizes real estate that is present within the delivery device. In addition, a secondary function of housing 102 eliminates the need for additional real estate and components. In other words, using top enclosure 104 as part of the reservoir maximizes available volume for drug storage in wearable drug delivery devices where reduced size is an important usability feature. Low reservoir pressure is accomplished at full fill volume to avoid reservoir pressure leading to drug delivery.

As described above, reservoir filling is accomplished through a septum in the top part of enclosure 104 that communicates with hole 110 into the reservoir itself. In one example, a septum pillar has a small undercut opening to the reservoir section. The flexible wall (film material) that defines the pouch is made from a film material as known to those skilled in the art or alternatively silicone or TPE or other material known to those skilled in the art that is flexible and can shape set.

The thin film (material) of the reservoir is preformed into the shape of the pocket in housing 102 giving a face-to-face placement between the two part enclosures. This eliminates any potential lost/dead volume in the reservoir. During filling, the only pressure required is to open the thin plastic film (as an example material) from housing 102. Once the film is opened, it will deflect into its mirrored shape from it unfilled state. This means that no residual pressure is present in the pouch as there is sufficient volume available that deflection or stressing of the pouch itself is not required.

In one example, the reservoir is configured to receive and store insulin for its delivery over a course of about three days, or as needed. However, reservoir size may be configured for storing any quantity of fluid as required.

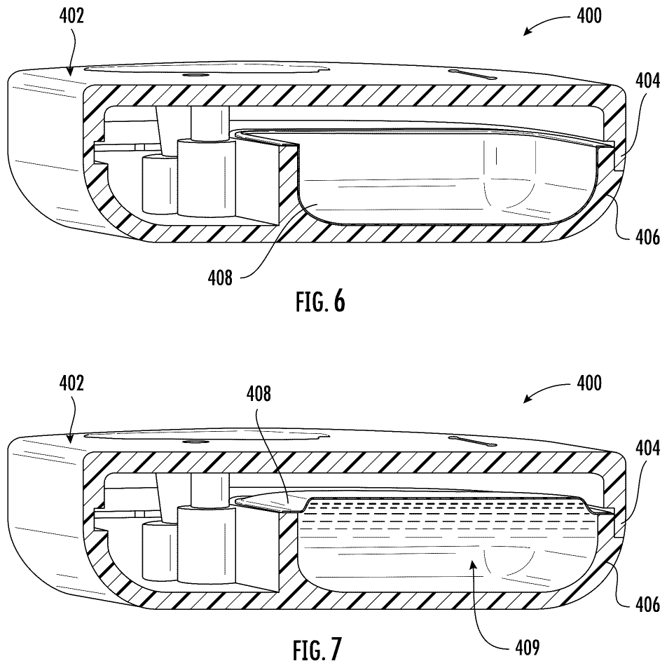

Reference is made to FIGS. 4-7 wherein various views of device 400 for delivering insulin or other medication is shown. Device 400 incorporates top housing 402 and top enclosure 404 and bottom enclosure 406 similar to the example device 100 above. Top enclosure 404 includes tray 404-1 with ledge 404-1a that extends around the periphery of tray 104-1. Ledge 104-1a defines the outer contour of an opening of tray 404-1 leading to a trough or cavity as shown. In this example device, tray 404-1 is rectangular and extends only in part across the width of top enclosure 104.

Device 100 further includes a film 408 that is joined to ledge 404-1a similar to the example device 100 above and formed to the contours of a wall of tray 404-1 that define the trough. That is, film 408 is thus fully line to line formed against the wall which removes any air that may be trapped between film 408 and wall of tray 404-1. Tray 404-1 and film 408 together define reservoir 409 for receiving and storing insulin or other medication. FIG. 7 depicts reservoir 409 fully filled with insulin. Tray 404-1 has an opening or hole 410 to enable a user to fill the reservoir and expand film 408 as reservoir is filled. Fill port 412 communicates with hole 410 and such port 412 is used to fill reservoir 409. Fill port 412 leakage is contained using a stopper or other mechanism. A syringe (not shown) may be used to puncture (to be inserted into) the stopper to access hole 410 to fill the reservoir 409. Tray 404-1 also has a release hole 414 that enables the release and delivery of insulin or other medication from the reservoir through outlet port 416.

A plastic film 408 (material) is used to define a pouch (reservoir 409) with the inner wall of top enclosure 404. In this example, the housing is split below a film material heat stake point to reduce complexity of the heat-staking process and to maximize space usage within the delivery device. Top enclosure 404 is injected molded.

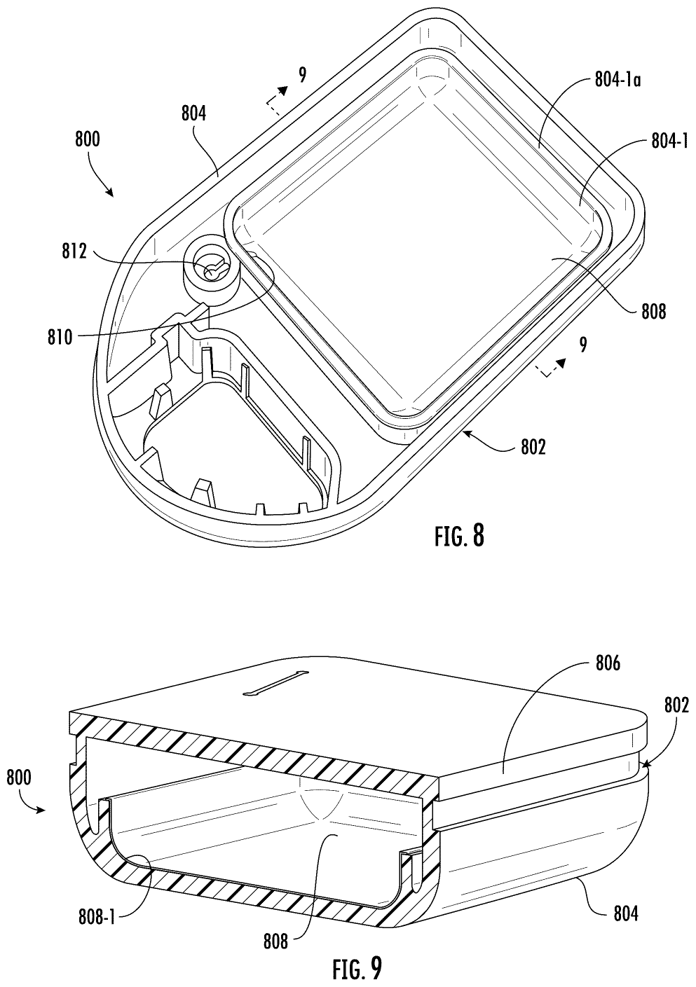

Reference is made to FIGS. 8 and 9 wherein two views of another example device 800 for delivering insulin is shown. Device 800 includes housing 802 with top and bottom enclosures 804 and 806. In this example, the reservoir is inset into the housing. Device 800 incorporates top housing 802, top enclosure 804 and bottom enclosure 806 similar to the example device 100 above. Top enclosure 804 includes tray 804-1 with ledge 804-1a that extends around the periphery of tray 804-1. Ledge 804-1a defines the outer contour of an opening of tray 804-1 leading to a trough or cavity as shown. In this example device, tray 804-1 is rectangular and extends only in part across the width of top enclosure 804.

Device 800 further includes a film 808 that is joined to ledge 804-1a similar to the example device 100 above and formed to the contours of a wall of tray 804-1 that define the trough. That is, film 808 is thus fully line to line formed against the wall which removes any air that may be trapped between film 808 and wall of tray 804-1. Tray 804-1 and film 808 together define reservoir for receiving and storing insulin or other medication. Tray 804-1 has an opening or hole 810 to enable a user to fill the reservoir and expand film 808 as reservoir is filled. Fill port 812 communicates with hole 810 and such port 812 is used to fill the reservoir. Fill port 812 leakage is contained using a stopper or other mechanism. A syringe (not shown) may be used to puncture (to be inserted into) the stopper to access hole 810 to fill the reservoir. Tray 804-1 also has a release hole (not shown) that enables the release and delivery of insulin or other medication from the reservoir.

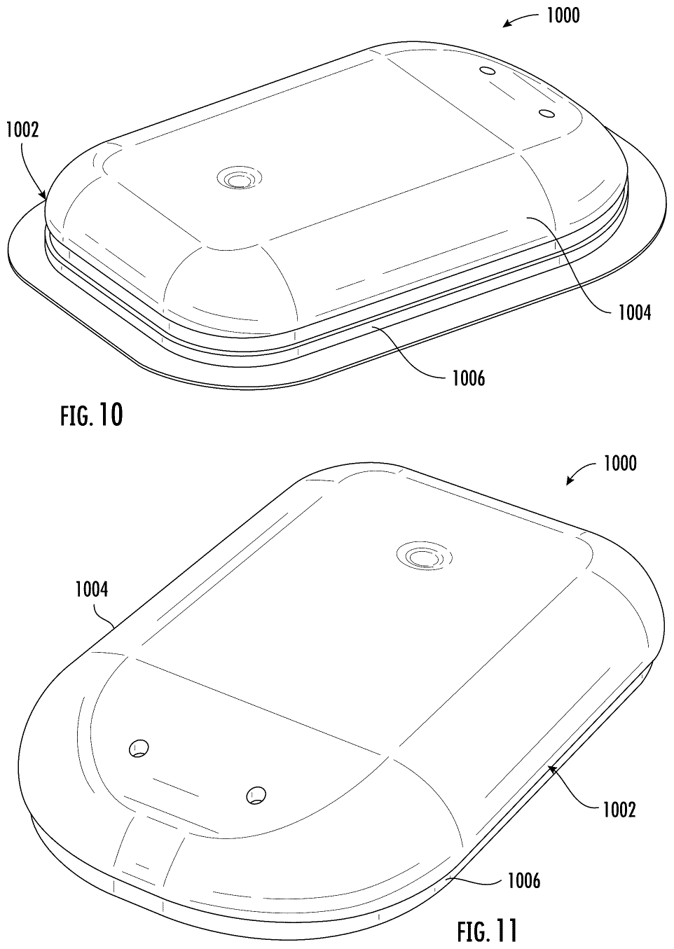

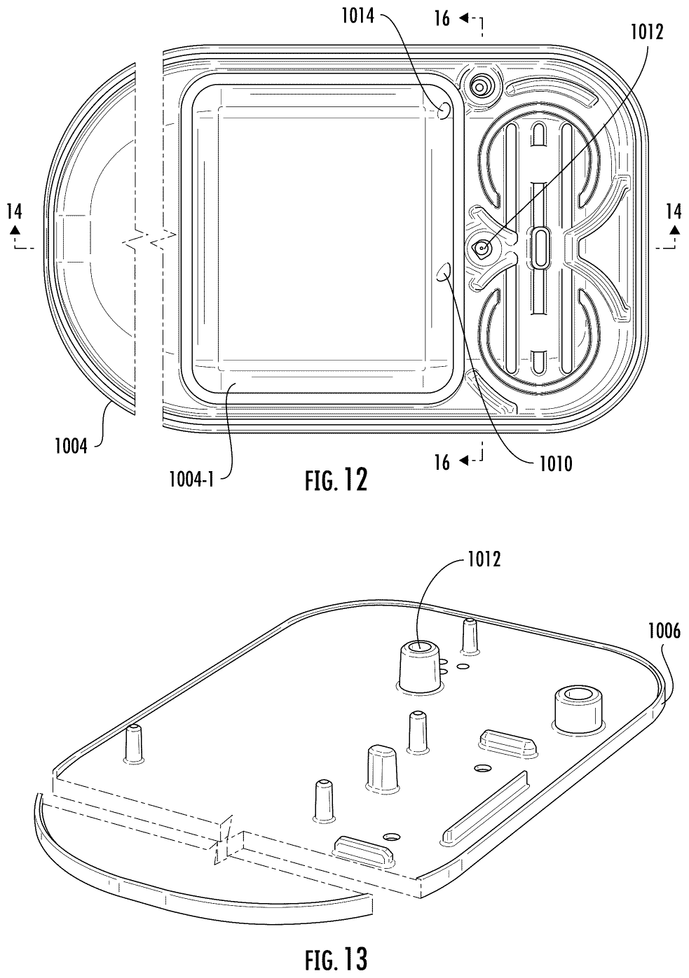

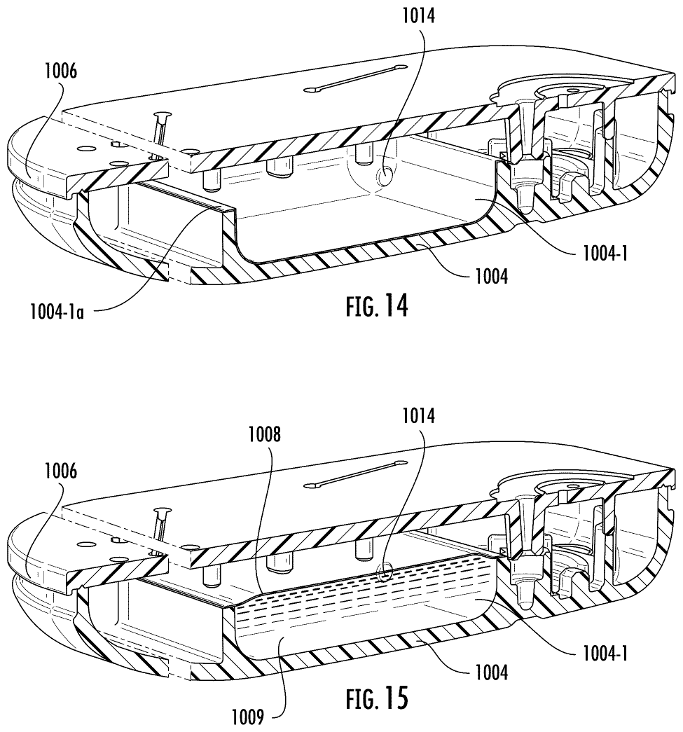

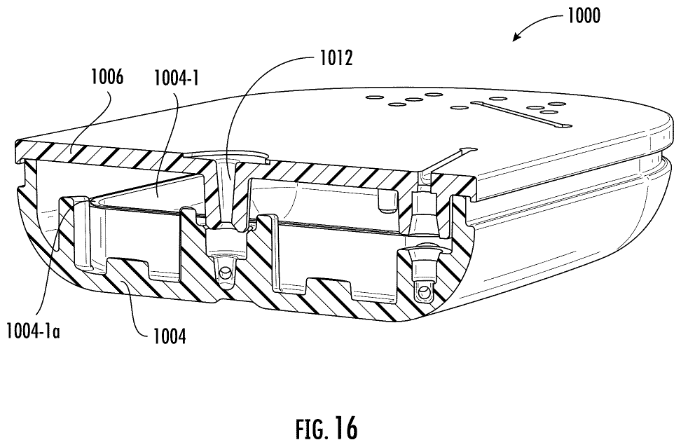

FIGS. 10-16 depicts another example device 1000 for delivering insulin or other medication. Device 1000 incorporates housing 1002 with top enclosure 1004 and bottom enclosure 1006. In this example, the reservoir is inset into the housing. Device 1000 incorporates top housing 1002 and top enclosure 1004 and bottom enclosure 1006 similar to the example device 100 above. Top enclosure 1004 includes tray 1004-1 with ledge 1004-1a that extends around the periphery of tray 1004-1. Ledge 1004-1a defines the outer contour of an opening of tray 1004-1 leading to a trough or cavity as shown. In this example device, tray 1004-1 is rectangular and extends across the width of top enclosure 1004.

Device 1000 further includes a film 1008 that is joined to ledge 1004-1a similar to the example device 100 above and formed to the contours of a wall of tray 1004-1 that define the trough. That is, film 1008 is thus fully line to line formed against the wall which removes any air that may be trapped between film 1008 and wall of tray 1004-1. Tray 1004-1 and film 1008 together define reservoir for receiving and storing insulin or other medication. Tray 1004-1 has an opening or hole 1010 to enable a user to fill reservoir 1009 and expand film 1008 as reservoir 1009 is filled. Fill port 1012 communicates with hole 1010 and such port 1012 is used to fill the reservoir. Fill port 1012 leakage is contained using a stopper or other mechanism. A syringe (not shown) may be used to puncture (to be inserted into) the stopper to access hole 1010 to fill reservoir 1009. Tray 1004-1 also has a release hole 1014 that enables the release and delivery of insulin or other medication from reservoir 1009.

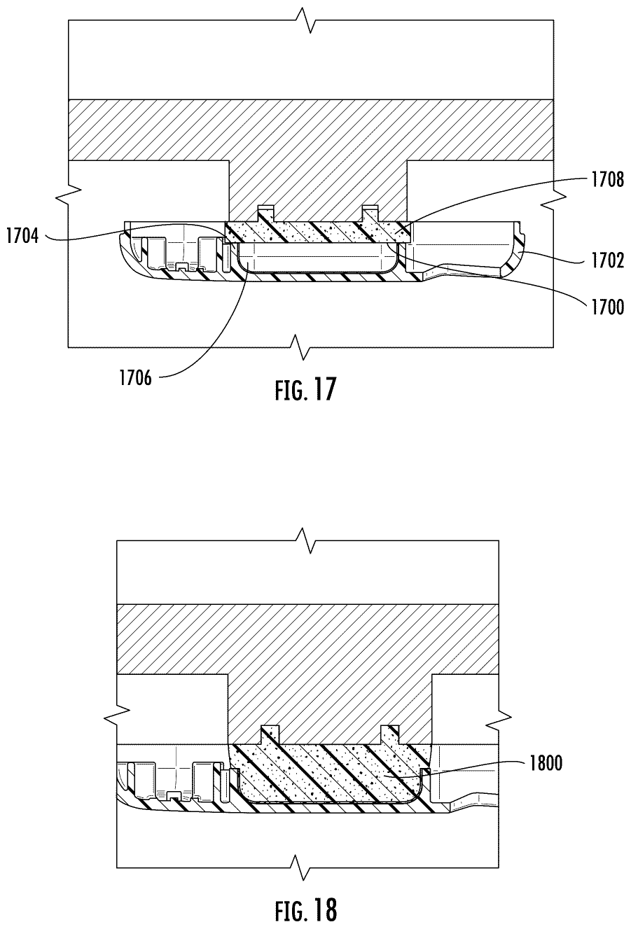

FIGS. 17 and 18 depict a cross-sectional views machinery used for the steps for joining a film 1700 to top enclosure 1702 of a device for delivering insulin. In particular, a film 1700 is joined to the housing, i.e., top enclosure 1702 by melting film 1700 to ledge 1704 of tray 1706 of top enclosure 1702 using a heat-staking process. A silicon heat stake block 1708 is used to perform the heat-staking process as shown in FIG. 17.

Film 1700 now bonded to housing is formed to tray 1706. This is done at a lower temperature where film 1700 material can flow and set without fully bonding film 1700 to the housing. This process again is done with a silicone heat forming tool (block 1800) as shown in FIG. 18. The compliance of the silicone allows for a full line-line forming of film 1700 against the walls of tray 1706 of the housing. This removes any air that may be trapped between the reservoir film 1700 and wall of tray 1706.

In summary, the reservoir as integrated into the example devices described hereinabove and method of making the same provide a drug storage for a wearable deliver device addresses a number of design needs while utilizing the least amount of space. This design uses the geometry of the housing as one side of the reservoir and a single thin film as the corresponding other part. Elimination of dead volume or air entrapment in the reservoir is achieved. Air can impede delivery and reduce dose accuracy. Further, low reservoir pressure at full fill volume is achieved to avoid reservoir pressure leading to medication delivery. The only pressure required is to open the thin plastic film from the housing. Once the film is opened it will deflect into its mirrored shape from its unfilled state. This means that no residual pressure is present in the pouch as there is sufficient volume available that deflection or stressing of the pouch itself is not required. Further this design allows for safe and effective drug storage that reduces size of the device for delivering insulin and allows more discretion for the users.

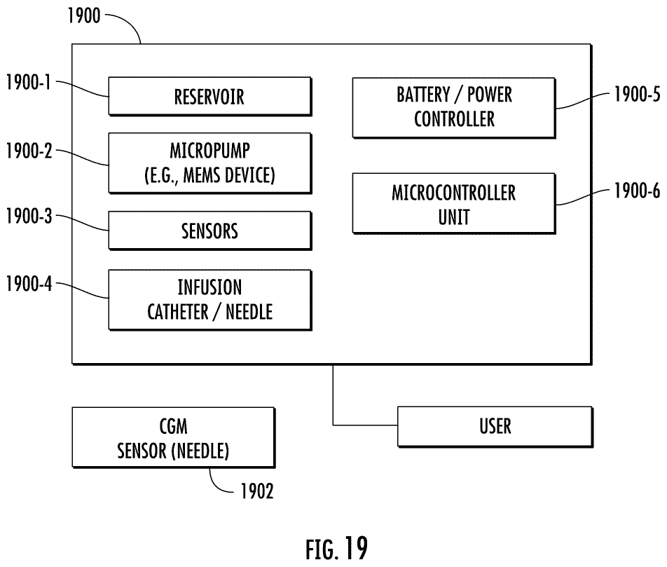

FIG. 19 depicts a box diagram of components of an example device for delivering medication of a diabetes management system. (Device 1900 is an example device described herein, e.g., represented as device 100 in FIG. 1.) Specifically, device 1900 incorporates several components or modules (not shown) in the fluidic pathway including reservoir 1900-1 for storing the insulin, micropump 1900-2 (as described hereinabove) for pumping the insulin or other medication, sensors 1900-3 (e.g., pressure) for sensing various parameters in the system and user and tubing connecting infusion catheter or infusion needle 1900-4 to reservoir 1900-1. Device 1900 also includes microcontroller unit (MCU) 1900-6 and battery and power controller 1900-5. MCU 1900-6 controls the operation of micropump 1900-2.

Device 1900 further includes CGM sensor 1902. CGM sensor 1902 is part of a diabetes management system. CGM sensor 1902 is shown as separate from the device 1900, but alternatively, it may be part of device 1900 in other examples. CGM or continuous glucose monitoring, as known to those skilled in the art, tracks user glucose levels and permits those levels to be used in algorithms that control flow rate. Infusion needle 1900-4 and CGM sensor (needle) 1902 are shown as separate components in FIG. 19. Alternatively, infusion catheter or needle 1900-4 and CGM sensor 1902 may be integrated in other examples within device 1900.

Reservoir 1900-1 is configured to receive and store insulin (or other medication) for its delivery over a course of about three days, or as needed. However, reservoir size may be configured for storing any quantity of fluid as required.

MCU 1900-5 electronically communicates with sensors 1900-3 and micropump 1900-2 as well as the CGM sensor 1900-6, as the monitoring components. Among several functions, MCU 1900-5 operates to control the operation of micropump 1900-2 to deliver insulin through infusion catheter or infusion needle 1900-7 from reservoir 1900-1 at specific doses, i.e., flow rates over specified time intervals, based on CGM data converted to desired flow rate via control algorithms.

Battery and power controller 1900-4 controls the power to MCU 1900-5 and micropump 1900-2 to enable those components to function properly as known to those skilled in the art.

FIG. 19 depict device 1900 with only a few components. Those skilled in the art know that device 1900 include additional components.

It is to be understood that the disclosure teaches examples of the illustrative embodiments and that many variations of the invention can easily be devised by those skilled in the art after reading this disclosure and that the scope of the present invention is to be determined by the claims below.

What is claimed is:

1. A device for delivering medication to a user, the device comprising:
first and second enclosures that together form a housing of the device, the housing configured to house a reservoir for storing the medication, wherein the first enclosure includes an inner wall of the housing; and
a film joined to the inner wall of the first enclosure, wherein the film and the inner wall of the first enclosure are configured to form walls of the reservoir for storing the medication.

2. The device of claim 1 wherein the first enclosure incudes a tray having a cavity and a ledge that extends around a periphery of the tray defining an opening that communicates with the cavity.

3. The device of claim 2 wherein the tray is rectangular and extend across the full width of the inner wall of the first enclosure.

4. The device of claim 1 wherein the film is plastic.

5. The device of claim 1 wherein the film is formed against the inner wall of the first enclosure thereby removing any air that may be trapped between film but enabling the film to expand as the medication fills the reservoir.

6. The device of claim 1 wherein the film joined to the inner wall of the first enclosure by bonding.

7. The device of claim 1 wherein the medication is insulin.

8. A device for delivering medication to a user, the device comprising:
first and second enclosures that together form a housing of the device;
a reservoir integrated into the housing configured to store the medication;
wherein the first enclosure includes an inner wall and a tray along the inner wall of the first enclosure; and
a film joined to the tray, wherein the film and the tray are configured to form walls of the reservoir.

9. The device of claim 8 wherein the tray includes a cavity and a ledge that extends around a periphery of the tray defining an opening that communicates with the cavity.

10. The device of claim 8 wherein the tray is rectangular and extend across the full width of the inner wall of the first enclosure.

11. The device of claim 8 wherein the film is plastic.

12. The device of claim 8 wherein the medication is insulin.

13. A method of integrating a reservoir into a housing of a device for delivering medication, wherein the housing includes a reservoir section for the reservoir, the method comprising:
joining a film to the reservoir section of the housing; and
forming the film against a wall of the reservoir section of the housing to thereby remove any air trapped between the film and wall of the reservoir.

14. The method of claim 13 wherein the reservoir section is a tray along a first enclosure of the housing, the tray having a ledge for joining the film to the ledge.

15. The method of claim 13 wherein the joining is done using heat-staking that melts materials of the film and housing.

16. The method of claim 13 further comprising joining a first enclosure and a second enclosure together to form the housing.

17. The method of claim 13 wherein the forming is performed using heat at a lower temperature so that the film is formed against the wall of the housing but enable the film to disengage from the wall as the reservoir is filled.

18. The method of claim 17 further comprising filling the reservoir.
